(19) United States
(12) Patent Application Publication
(10) Pub. No.: US 2025/0277778 A1
(43) Pub. Date: Sep. 4, 2025

(54) QUANTITATIVE IDENTIFICATION METHOD FOR PROVENANCE OF CLASTIC ROCKS

(71) Applicants: Southwest Petroleum University, Chengdu (CN); China University of Petroleum (Beijing), Beijing (CN); China University of Geosciences ( Wuhan), Wuhan (CN)

(72) Inventors: Xinyang He, Chengdu (CN); Kun Zhang, Chengdu (CN); Zhenxue Jiang, Beijing (CN); Yan Song, Beijing (CN); Shu Jiang, Wuhan (CN); Hulin Niu, Chengdu (CN); Chengzao Jia, Beijing (CN); Lin Jiang, Beijing (CN); Xueying Wang, Chengdu (CN); Xiong Ding, Chengdu (CN); Yi Shu, Wuhan (CN); Yi Zhang, Chengdu (CN); Yiting Qiao, Chengdu (CN); Jiayi Liu, Chengdu (CN); Jun Peng, Chengdu (CN); Bin Li, Chengdu (CN); Jinhua Liu, Chengdu (CN); Binsong Zheng, Chengdu (CN); Lei Chen, Chengdu (CN); Xuefei Yang, Chengdu (CN); Fengli Han, Chengdu (CN); Weishi Tang, Chengdu (CN); Jingru Ruan, Chengdu (CN); Hengfeng Gou, Chengdu (CN); Yi Xiao, Chengdu (CN); Lintao Li, Chengdu (CN); Yipeng Liu, Chengdu (CN); Ping Liu, Chengdu (CN); Meijia Wu, Chengdu (CN); Lu Lu, Chengdu (CN); Zeyun Wang, Chengdu (CN); Laiting Ye, Chengdu (CN)

(21) Appl. No.: 19/000,057

(22) Filed: Dec. 23, 2024

(30) Foreign Application Priority Data
Feb. 29, 2024 (CN) 2024102257803

(51) Int. Cl. G01N 33/24; H01J 49/10

(52) U.S. Cl. CPC G01N 33/24; H01J 49/105

(57) ABSTRACT

A quantitative identification method for provenance of clastic rocks is provided, which includes: determining a study area, a target horizon of the study area and potential provenances of the target horizon, and performing sampling on known wells of the target horizon and the potential provenances to obtain rock samples; measuring contents of light minerals, heavy minerals, and trace elements to obtain measurement results of the known wells and measurement results of the potential provenances, establishing standard fuzzy sets based on the measurement results of the potential provenances, and establishing to-be-identified fuzzy sets based on the measurement results of the known wells; assigning weight coefficients to three indicators; calculating weighted closenesses between the to-be-identified fuzzy sets and the standard fuzzy sets according to the weight coefficients; and determining a provenance of the target horizon according to the weighted closenesses and a principle of proximity selection.

### CROSS-REFERENCE TO RELATED APPLICATION

This application claims priority to Chinese Patent Application No. 202410225780.3, filed on Feb. 29, 2024, which is herein incorporated by reference in its entirety.

### TECHNICAL FIELD

The disclosure relates to the technical field of provenance analysis, particularly to a quantitative identification method for provenance of a clastic rock.

### BACKGROUND

A provenance refers to a supply zone of clastic materials in a sedimentary basin. Provenance analysis is a crucial step in reconstructing paleoclimate, paleoenvironment, and paleogeographic positions, and serves as the foundation for regional paleoclimate and paleoenvironment reconstruction, as well as paleotectonic background interpretation. Through the provenance analysis, one can not only understand the formation history of the provenance but also, based on stable isotope dating, restore missing geological epochs in the provenance source. With the diversification of modern testing technologies and the continuous improvement of methodological standards, types of methods of the provenance analysis have increased and matured over time. Currently, several widely used analysis methods primarily include a sedimentological analysis method, a petrological method, a geochronological method, and a paleocurrent direction indication method.

The sedimentological analysis method conducts stratigraphic correlation and division based on existing drilling, well logging, and seismic data in a study area, compiles isopach maps of stratigraphic thickness and conglomerate thickness, and infers relative positions of provenances of the study area. The sedimentological analysis method involves extensive data statistics and can only roughly determine directions of the provenances, but cannot determine specific locations of the provenances and properties of a parent rock.

The petrological method infers types of parent rocks in a provenance according to the combination of terrigenous clastic from the parent rocks of terrigenous detrital rocks in a study area. The petrological method involves extensive data statistics, and in an actual sedimentary environment, sediments often come from multiple provenances, which may have different types of parent rocks. Therefore, there is uncertainty in the inference of provenances by the petrological method.

The geochronological method uses single-grain clastic mineral isotope dating to analyze provenance. Currently applied methods of the geochronological method mainly include fission track dating of elastic grains (apatite, zircon), and U-Pb dating of U-bearing minerals (zircon, monazite, and sphene). This method is economically costly, and a U-Pb system of clastic grains may be disturbed during later thermal events, leading to inaccurate age data. When processing and interpreting zircon U-Pb age data, researchers may need to set thresholds to determine the concordance of ages, which introduces a degree of subjectivity.

The paleocurrent direction indication method determines transport directions of sediments through measuring directions of paleocurrents, and thereby determines provenance of ancient provenances. However, determining the directions of the paleocurrents usually relies on the analysis of sedimentary structures, such as cross-bedding and ripple marks, these sedimentary structures may be influenced by various factors during deposition, such as sedimentation rate, water flow intensity, and weathering, which may lead to misjudgment of the directions of the paleocurrents and subsequently affect the accuracy of provenance identification.

It can be seen that existing techniques primarily rely on single methods and qualitative judgments, with lower accuracy. Therefore, there is an urgent need for a method that can accurately perform quantitative analysis of provenance.

### SUMMARY

In view of the above problems, the disclosure provides a quantitative identification method for provenance of a clastic rock.

Technical solutions of the disclosure are as follows.

A quantitative identification method for provenance of clastic rocks includes the following steps:
S1, determining a study area, a target horizon of the study area and potential provenances of the target horizon, and performing sampling on known wells of the target horizon and the potential provenances to obtain rock samples;
S2, measuring contents of light minerals, heavy minerals, and trace elements in the rock samples to obtain measurement results of the known wells and measurement results of the potential provenances, establishing standard fuzzy sets based on the measurement results of the potential provenances, and establishing to-be-identified fuzzy sets based on the measurement results of the known wells;
S3, assigning weight coefficients related to importance in provenance discrimination to three indicators respectively corresponding to the light minerals, the heavy minerals and the trace elements;
S4, calculating weighted closenesses between the to-be-identified fuzzy sets and the standard fuzzy sets according to the weight coefficients; and
S5, determining a provenance of the target horizon according to the weighted closenesses and a principle of proximity selection.

In an embodiment, the measuring contents of light minerals, heavy minerals, and trace elements in the rock samples in step S2 includes:
performing thin-section preparation and microscopic identification on the rock samples to thereby obtain the contents of the light minerals and the contents of the heavy minerals; and
performing content analysis on the trace elements by plasma mass spectrometry, to thereby obtain the contents of the trace elements.

In an embodiment, in step S2, the light minerals include quartz, feldspar, rock debris, and other light minerals, and the rock debris includes igneous rock debris, metamorphic rock debris, and sedimentary rock debris; the heavy minerals include heavy mineral assemblage, epidote, garnet, sphene, titanomagnetite and white titanium ore, and the heavy mineral assemblage consists of rutile, zircon and tourmaline; and the trace elements include scandium (Sc), vanadium (V), chromium (Cr), cobalt (Co), nickel (Ni), zinc (Zn), gallium (Ga), rubidium (Rb) and zirconium (Zr).

In an embodiment, the assigning weight coefficients related to importance in provenance discrimination to three indicators respectively corresponding to the light minerals, the heavy minerals and the trace elements in step S3 includes using a pairwise comparison method to assign the weight coefficients to the three indicators.

In an embodiment, the using a pairwise comparison method to assign the weight coefficients to the three indicators includes:
step S31, based on geological experience, performing pairwise comparison on importance in provenance discrimination of the three indicators respectively corresponding to the light minerals, the heavy minerals, and the trace elements, to thereby construct a judgment matrix:

$$A = ( \begin{matrix}1 & \frac{1}{5} & \frac{1}{3} \\ 5 & 1 & 3 \\ 3 & \frac{1}{3} & 1\end{matrix} ) ; \qquad ( 1 )$$

step S32, normalizing each column of the judgment matrix to obtain a a first matrix A₁, expressed as follows:

$${A}_{1} = ( \begin{matrix}\frac{1}{9} & \frac{3}{23} & \frac{1}{13} \\ \frac{5}{9} & \frac{15}{23} & \frac{9}{23} \\ \frac{3}{9} & \frac{5}{23} & \frac{3}{13}\end{matrix} ) ; \qquad ( 2 )$$

step S33, performing a summation operation on the first matrix A₁ to obtain a second matrix A₂, expressed as follows:

$${A}_{2} = ( \begin{matrix}\begin{matrix}\frac{857}{2691} \\ \frac{5113}{2691}\end{matrix} \\ \frac{2103}{2691}\end{matrix} ) ; \qquad ( 3 )$$

and
step S34, normalizing the second matrix A₂ to obtain a third matrix A₃, expressed as follows:

$${A}_{3} = ( \begin{matrix}0.106 \\ 0.634 \\ 0.26\end{matrix} ) , \qquad ( 4 )$$

and
based on the third matrix A₃, determining the weight coefficients related to importance in provenance discrimination for the three indicators to be 0.106 for the light minerals, 0.634 for the heavy minerals, and 0.260 for the trace elements.

In an embodiment, in step S4, each of the weighted closenesses is calculated through the following formula:

$$σ ( A , B ) {=}^{def} \frac{{\sum }_{k = 1}^{n} [ A ( {x}_{k} ) ∧ B ( {x}_{k} ) ]}{{\sum }_{k = 1}^{n} [ A ( {x}_{k} ) ∨ B ( {x}_{k} ) ]} \times q \qquad ( 5 )$$

where σ(A, B) represents a weighted closeness between a set A and a set B; the set A and the set B represent a to-be-identified fuzzy set and a standard fuzzy set, respectively; n represents a total number of elements in each of the to-be-identified fuzzy set and the standard fuzzy set; xₖ represents a k-th element in a corresponding fuzzy set; ∧ represents taking an infimum when two related elements are compared; ∨ represents taking a supremum when two related elements are compared; and q represents a corresponding weight coefficient.

In an embodiment, when the provenance of the target horizon comprises multiple provenances, the quantitative identification method for provenance of a clastic rock further includes:
step S6, summing weighted closenesses of light minerals, heavy minerals and trace elements of each provenance of the multiple provenances to obtain a comprehensive weighted closeness of each provenance of the multiple provenances; and
step S7, determining a primary provenance, a relatively primary provenance and a secondary provenance of the target horizon according to the comprehensive weighted closeness of each provenance of the multiple provenances and the principle of proximity selection.

The disclosure at least has the following beneficial effects.

The disclosure integrates the characteristics of heavy mineral content, light mineral content, and trace element content in rocks, combined with a fuzzy recognition method, to enable quantitative analysis of provenance and quantitative discrimination of the provenance direction of clastic rocks. It aids in reservoir sandstone body prediction, which is conducive to guiding the next step in oil and gas exploration deployment.

Further, in an embodiment, after the provenance of the target horizon is determined, the quantitative identification method for provenance of a clastic rock further includes: in response to a ratio of sedimentary rock debris of a parent rock of the provenance being greater than a preset threshold, exploring and developing the target horizon. Specifically, the parent rock consists of the sedimentary rock debris, igneous rock debris, and metamorphic rock debris. A higher proportion of the sedimentary rock debris is more conducive to the development of pores in sandstone, which is beneficial for the formation of good sandstone oil and gas reservoirs. The corresponding area, i.e., the target horizon, is prioritized for exploration and development. Conversely, the igneous rock debris and the metamorphic rock debris are difficult to dissolve later on, and can block primary pores and throats, which is detrimental to pore development. Therefore, the target horizon with a higher proportion of igneous and metamorphic rock debris tend to form reservoirs with poorer storage capacity.

### BRIEF DESCRIPTION OF DRAWINGS

To clearly illustrate the technical solutions in the embodiments of the disclosure or in the related art, the following provides a brief introduction to accompanying drawings required for describing the embodiments or the related art. It is evident that the accompanying drawings described below are merely some embodiments of the disclosure. For those of ordinary skill in the art, other drawings may be obtained based on these accompanying drawings without exercising creative efforts.

FIG. 1 illustrates a schematic diagram showing location distribution of potential provenances and a study area according to an embodiment of the disclosure.

FIG. 2 illustrates a schematic diagram showing planar distribution of main provenances in a study area according to an embodiment of the disclosure.

### DETAILED DESCRIPTION OF EMBODIMENTS

The disclosure will be further explained with the accompanying drawings and embodiments. It should be noted that the embodiments in this application and the technical features in the embodiments can be combined with each other without conflict. It should be pointed out that unless otherwise specified, all technical and scientific terms used in this application have the same meaning as commonly understood by ordinary technicians in the technical field to which this application belongs. Similar words such as “including” or “containing” used in the disclosure mean that elements or objects appearing before the words cover the elements or objects listed after the words and equivalents of the elements or objects, without excluding other elements or objects.

An embodiment of the disclosure provides a quantitative identification method for provenance of clastic rocks, which includes steps S1-S7.

In step S1, a study area, a target horizon of the study area and potential provenances of the target horizon are determined, and sampling is performed on known wells of the target horizon and the potential provenances to obtain rock samples.

It should be noted that each potential provenance is a peripheral paleocontinent of the study area, and a determination method for the peripheral paleocontinent is the prior art well known in the art, which will not be described in detail herein.

In step S2, contents of light minerals, heavy minerals, and trace elements in the rock samples are measured to obtain measurement results of the known wells and measurement results of the potential provenances; standard fuzzy sets are established based on the measurement results of the potential provenances; and to-be-identified fuzzy sets are established based on the measurement results of the known wells.

In a specific embodiment, the rock samples are subjected to thin-section preparation and microscopic identification, to thereby measure the contents of the light minerals and the heavy minerals. Content analysis of the trace elements is performed by plasma mass spectrometry to thereby obtain the contents of the trace elements. It should be noted that the measurement of contents is existing technology. In addition to the method adopted in this embodiment, other methods of existing technology that can obtain the contents of the light minerals, the heavy minerals, and the trace elements are also applicable to the disclosure.

In a specific embodiment, the light minerals include quartz, feldspar, rock debris, and other light minerals. The rock debris includes igneous rock debris, metamorphic rock debris, and sedimentary rock debris.

The heavy minerals include heavy mineral assemblage, epidote, garnet, sphene, titanomagnetite and white titanium ore, and the heavy mineral assemblage consists of rutile, zircon and tourmaline.

The trace elements include scandium (Sc), vanadium (V), chromium (Cr), cobalt (Co), nickel (Ni), zinc (Zn), gallium (Ga), rubidium (Rb) and zirconium (Zr).

It should be noted that in the above embodiments, the other light minerals refer to all other light minerals except the listed light minerals, i.e., the quartz, the feldspar and the rock debris.

In step S3, weight coefficients related to importance in provenance discrimination are assigned to three indicators respectively corresponding to the light minerals, the heavy minerals and the trace elements.

In a specific embodiment, a pairwise comparison method is used to assign the weight coefficients, and the step S3 specifically includes step S31-S37.

In step S31, based on geological experience, pairwise comparison is performed on importance in provenance discrimination of the three indicators respectively corresponding to the light minerals, the heavy minerals, and the trace elements, and thus a judgment matrix is constructed as follows:

$$A = ( \begin{matrix}1 & \frac{1}{5} & \frac{1}{3} \\ 5 & 1 & 3 \\ 3 & \frac{1}{3} & 1\end{matrix} ) . \qquad ( 1 )$$

In step S32, each column of the judgment matrix is normalized to obtain a first matrix A₁, expressed as follows:

$${A}_{1} = ( \begin{matrix}\frac{1}{9} & \frac{3}{23} & \frac{1}{13} \\ \frac{5}{9} & \frac{15}{23} & \frac{9}{23} \\ \frac{3}{9} & \frac{5}{23} & \frac{3}{13}\end{matrix} ) . \qquad ( 2 )$$

In step S33, a summation operation is performed on the first matrix A₁ to obtain a second matrix A₂, expressed as follows:

$${A}_{2} = ( \begin{matrix}\frac{857}{2691} \\ \frac{5113}{2691} \\ \frac{2103}{2691}\end{matrix} ) . \qquad ( 3 )$$

In step S34, the second matrix A₂ is normalized to obtain a third matrix A₃, expressed as follows, and based on the third matrix A₃, the weight coefficients related to importance in provenance discrimination for the three indicators are determined to be 0.106 for the light minerals, 0.634 for the heavy minerals, and 0.260 for the trace elements:

$${A}_{3} = ( \begin{matrix}0.106 \\ 0.634 \\ 0.26\end{matrix} ) , \qquad ( 4 )$$

and based on the third matrix A₃, the weight coefficients related to importance in provenance discrimination for the three indicators are determined to be 0.106 for the light minerals, 0.634 for the heavy minerals, and 0.260 for the trace elements.

It should be noted that when the weight coefficients are assigned using the pairwise comparison method, in addition to using the judgment matrix constructed in the step S31, other importance values can also be used to construct the judgment matrix. The judgment matrix is denoted as A=(aᵢⱼ)_{m×n}, and common values of an element aᵢⱼ are shown in Table 1, Bᵢ and Bᵢ respectively correspond to two of the three indicators respectively corresponding to the light minerals, the heavy minerals, and the trace elements.

TABLE 1
Pairwise relative importance value table

| | Bᵢ vs Bⱼ | | | | | | | | |
|---|---|---|---|---|---|---|---|---|---|
| | Slightly | | Very | Absolutely | | Slightly | | Very | Absolutely |
| | stronger | Strong | strong | strong | Same | weaker | Weak | weak | weak |
| aᵢⱼ | 3 | 5 | 7 | 9 | 1 | ⅓ | ⅕ | 1/7 | 1/9 |

In a specific embodiment, after determining the weight coefficients in the step 34, the quantitative identification method determining whether the weight coefficients are acceptable, which specifically includes steps S35-S37.

In step S35, a maximum eigenvalue λₘₐₓ of the judgement matrix is calculated through following formulas:

$$AW = ( \begin{matrix}1 & \frac{1}{5} & \frac{1}{3} \\ 5 & 1 & 3 \\ 3 & \frac{1}{3} & 1\end{matrix} ) \times ( \begin{matrix}0.106 \\ 0.634 \\ 0.26\end{matrix} ) = ( \begin{matrix}0.319 \\ 1.944 \\ 0.789\end{matrix} ) \qquad ( 6 )$$

$${λ}_{{max}^{=}} \frac{1}{m} {\sum }_{i = 1}^{m} \frac{{( AW )}_{i}}{{w}_{i}} = \frac{1}{3} ( \frac{0.319}{0.106} + \frac{1.944}{0.634} + \frac{0.789}{0.26} ) = 3.04 \qquad ( 7 )$$

where m represents an order of a matrix, (AW)ᵢ represents an i-th component of a matrix AW, and wᵢ represents an i-th component of a matrix W (i.e., the third matrix A₃).

In step S36, a consistency index of the judgment matrix is calculated through the following formula:

$$C = \frac{{λ}_{max} - m}{m - 1} = \frac{3.04 - 3}{3 - 1} = 0 \text{.02} . \qquad ( 8 )$$

In step S37, a consistency ratio of the judgment matrix is calculated through the following formula:

$${C}_{R} = \frac{C}{R} = \frac{0.02}{0.52} = 0.038 \qquad ( 9 )$$

where R represents a random consistency index, which can be found from Table 2.

TABLE 2
Random consistency index Rᵢ

| m | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|
| Rᵢ | 0 | 0 | 0.52 | 0.89 | 1.12 | 1.26 | 1.36 | 1.41 | 1.46 | 1.49 | 1.52 | 1.54 | 1.56 | 1.58 | 1.59 |

When the consistency ratio C_{R}<0.1, it is determined that the consistency of the judgment matrix is acceptable. In the above embodiment, C_{R}<0.1, therefore, the consistency of the judgment matrix of the above embodiment is acceptable, and the obtained weight coefficients are also acceptable.

In step S4, according to the weight coefficients, weighted closenesses between the to-be-identified fuzzy sets and the standard fuzzy sets are calculated.

In a specific embodiment, each of the weighted closenesses is calculated through the following formula:

$$σ ( A , B ) {=}^{def} \frac{{\sum }_{k = 1}^{n} [ A ( {x}_{k} ) ∧ B ( {x}_{k} ) ]}{{\sum }_{k = 1}^{n} [ A ( {x}_{k} ) ∨ B ( {x}_{k} ) ]} \times q \qquad ( 5 )$$

where σ(A,B) represents a weighted closeness between a set A and a set B; the set A and the set B represent a to-be-identified fuzzy set and a standard fuzzy set, respectively; n represents a total number of elements in each of the to-be-identified fuzzy set and the standard fuzzy set; xₖ represents a k-th element in a corresponding fuzzy set; ∧ represents taking an infimum (i.e., the greatest lower bound) when two elements are compared; ∨ represents taking a supremum (i.e., the least upper bound) when two elements are compared; and q represents a corresponding weight coefficient.

In step S5, according to the weighted closenesses and a principle of proximity selection, a provenance of the target horizon is determined.

It should be noted that determining the provenance of the target horizon according to the principle of proximity selection is selecting a potential provenance with the greatest weighted closeness of the potential provenances as the provenance of the target horizon.

In a specific embodiment, when the provenance of the target horizon includes multiple provenances, the quantitative identification method for provenance of a clastic rock further includes the following steps S6 and S7.

In step S6: weighted closenesses of light minerals, heavy minerals and trace elements of each provenance of the multiple provenances are summed to obtain a comprehensive weighted closeness of each provenance.

In step S7, a primary provenance, a relatively primary provenance and a secondary provenance of the target horizon are determined according to the comprehensive weighted closeness of each provenance and the principle of proximity selection.

In a specific embodiment, taking a lake basin E in northern China as an example, the quantitative identification method for provenance of a clastic rock is adopted to determine a provenance of a clastic rock of the lake basin E, and a specific process is as follows.

Firstly, rock samples of known wells and potential provenances in a target horizon are obtained.

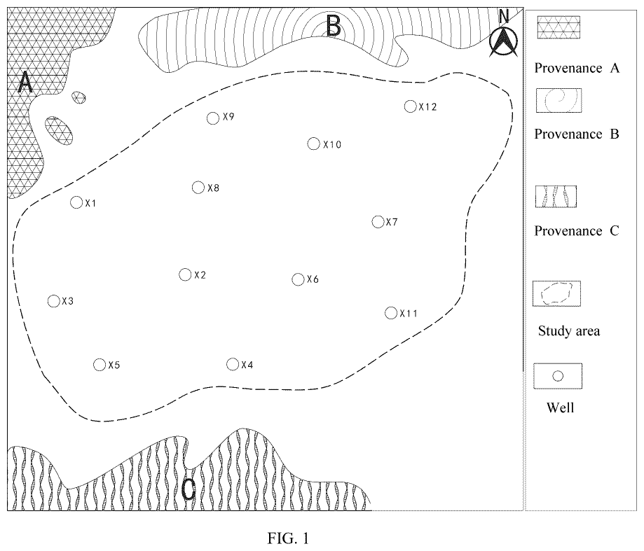

In this embodiment, as shown in FIG. 1, during the late Triassic sedimentary period, there were many paleocontinents around the lake basin E in northern China, which provided material sources for the sedimentation in the lake basin E. Through the investigation and analysis of regional data, it is found that the reliable paleocontinents (that is, potential provenances) in this lake basin E are: a paleocontinent A in the northwest, a paleocontinent B in the north and a paleocontinent C in the south.

In order determine material sources (i.e., provenance) of a horizon H (i.e., the target horizon) in the lake basin E, samples are uniformly collected from the paleocontinents A, B, C, and X1 to X12 wells of the horizon H of in the lake basin E (with no less than 10 samples for each paleocontinent and no less than 10 samples for each well), to thereby obtaining rock samples from each sampling site.

Secondly, contents of light minerals, heavy minerals, and trace elements in the rock samples are measured.

Specifically, the rock samples are subjected to thin-section preparation and microscopic identification, to thereby measure the contents of the light minerals and the heavy minerals. Content analysis of the trace elements is performed by plasma mass spectrometry to thereby obtain the contents of the trace elements. To avoid random errors, experimental results of different samples from the same potential provenance or the same well are averaged. Experimental results of the rock samples from the potential provenances A, B, and C, and the lake basin E are statistically processed and normalized to obtain data shown in Tables 3 to 5.

TABLE 3
Measurement result of contents of light minerals in Rock Samples

| | | | Rock debris (%) | | | |
|---|---|---|---|---|---|---|
| | | | igneous | metamorphic | sedimentary | |
| Sample | Quartz | Feldspar | rock | rock | rock | Other |
| source | (%) | (%) | debris | debris | debris | (%) |
| Provenance A | 30 | 35 | 20 | 10 | 1 | 4 |
| Provenance B | 37 | 41 | 10 | 5 | 0 | 7 |
| Provenance C | 23 | 47 | 15 | 13 | 1 | 1 |
| Well X1 | 30 | 32 | 19 | 10 | 2 | 7 |
| Well X2 | 31 | 39 | 17 | 5 | 0 | 8 |
| Well X3 | 27 | 44 | 21 | 3 | 1 | 4 |
| Well X4 | 25 | 49 | 13 | 12 | 0 | 1 |
| Well X5 | 22 | 45 | 17 | 16 | 0 | 0 |
| Well X6 | 22 | 47 | 10 | 15 | 1 | 5 |
| Well X7 | 36 | 40 | 7 | 14 | 2 | 1 |
| Well X8 | 29 | 38 | 16 | 7 | 10 | 0 |
| Well X9 | 32 | 42 | 7 | 15 | 0 | 4 |
| Well X10 | 25 | 35 | 25 | 12 | 0 | 3 |
| Well X11 | 24 | 46 | 14 | 13 | 0 | 3 |
| Well X12 | 36 | 42 | 9 | 5 | 0 | 8 |

TABLE 4
Measurement result of contents of heavy minerals in Rock Samples

| | Rutile + | | | | | White |
|---|---|---|---|---|---|---|
| | Zircon + | | | | | titanium |
| Sample | Tourmaline | Epidote | Garnet | Sphene | Titanomagnetite | ore |
| source | (%) | (%) | (%) | (%) | (%) | (%) |
| Provenance A | 40 | 5 | 15 | 0 | 40 | 0 |
| Provenance B | 28 | 0 | 45 | 0 | 25 | 2 |
| Provenance C | 36 | 0 | 26 | 0 | 38 | 0 |
| Well X1 | 42 | 5 | 12 | 1 | 39 | 1 |
| Well X2 | 35 | 3 | 20 | 0 | 28 | 14 |
| Well X3 | 17 | 0 | 54 | 1 | 20 | 8 |
| Well X4 | 39 | 5 | 32 | 2 | 20 | 2 |
| Well X5 | 36 | 4 | 23 | 3 | 32 | 2 |
| Well X6 | 33 | 2 | 51 | 0 | 14 | 0 |
| Well X7 | 48 | 0 | 37 | 0 | 14 | 1 |
| Well X8 | 32 | 1 | 41 | 1 | 20 | 5 |
| Well X9 | 26 | 1 | 42 | 0 | 26 | 5 |
| Well X10 | 20 | 2 | 56 | 0 | 21 | 1 |
| Well X11 | 35 | 1 | 27 | 1 | 35 | 1 |
| Well X12 | 29 | 1 | 43 | 0 | 26 | 1 |

TABLE 5
Measurement result of contents of trace elements in Rock Samples

| Sample | | | | | | | | | |
|---|---|---|---|---|---|---|---|---|---|
| source | Sc(%) | V(%) | Cr(%) | Co(%) | Ni(%) | Zn(%) | Ga(%) | Rb(%) | Zr(%) |
| Provenance A | 12 | 5 | 17 | 20 | 1 | 28 | 10 | 4 | 3 |
| Provenance B | 20 | 11 | 32 | 5 | 16 | 2 | 7 | 5 | 2 |
| Provenance C | 32 | 20 | 23 | 6 | 3 | 11 | 1 | 4 | 0 |
| Well X1 | 13 | 5 | 16 | 21 | 1 | 29 | 9 | 3 | 3 |
| Well X2 | 18 | 12 | 37 | 5 | 9 | 13 | 0 | 2 | 4 |
| Well X3 | 8 | 24 | 24 | 16 | 6 | 8 | 10 | 0 | 4 |
| Well X4 | 30 | 7 | 15 | 13 | 20 | 2 | 7 | 5 | 1 |
| Well X5 | 34 | 17 | 20 | 5 | 2 | 9 | 5 | 2 | 6 |
| Well X6 | 4 | 10 | 1 | 7 | 13 | 14 | 25 | 17 | 9 |
| Well X7 | 32 | 21 | 13 | 6 | 15 | 1 | 8 | 3 | 1 |
| Well X8 | 6 | 13 | 23 | 22 | 2 | 14 | 14 | 1 | 5 |
| Well X9 | 14 | 2 | 7 | 34 | 9 | 7 | 12 | 10 | 5 |
| Well X10 | 7 | 2 | 26 | 2 | 2 | 1 | 9 | 17 | 34 |
| Well X11 | 17 | 12 | 16 | 13 | 16 | 15 | 1 | 2 | 8 |
| Well X12 | 21 | 12 | 30 | 4 | 17 | 3 | 8 | 4 | 1 |

Thirdly, weight coefficients related to importance in provenance discrimination are assigned to three indicators respectively corresponding to the light minerals, the heavy minerals and the trace elements.

In this embodiment, according to the formulas (1)-(4), the weight coefficients related to importance in provenance discrimination for the three indicators are determined to be 0.106 for the light minerals, 0.634 for the heavy minerals, and 0.260 for the trace elements.

Fourthly, fuzzy identification is performed.

According to the measurement result in Table 3, let a universe U={light minerals}, which includes six indicators consisting of quartz, feldspar, igneous rock debris, metamorphic rock debris, sedimentary rock debris and other, and the six indicators form a standard fuzzy set {quartz, feldspar, igneous rock debris, metamorphic rock debris, sedimentary rock debris, others}. Then a standard fuzzy set of the provenance A on the universe U is: U_{A}={0.3, 0.35, 0.2, 0.1, 0.01, 0.04}; a standard fuzzy set of the provenance B on the universe U is U_{B}={0.37, 0.41, 0.1, 0.05, 0.07}; and a standard fuzzy set of the provenance C on the universe U is U_{C}={0.23, 0.47, 0.15, 0.13, 0.01, 0.01}.

For each well, there are six indexes, and a single well has a to-be-identified fuzzy set on the universe U. For example, a to-be-identified fuzzy set of the Well X1 on the universe U is: U_{X1}={0.3, 0.32, 0.19, 0.1, 0.02, 0.07}, then a weighted closeness between the to-be-identified fuzzy set U_{X1} and the standard fuzzy set U_{A} is expressed as follows:

$$σ ( {U}_{X 1} , {U}_{A} ) = \frac{\begin{matrix} \\ [ ( 0.3 ∧ 0.3 ) + ( 0.32 ∧ 0.35 ) + \\ ( 0.19 ∧ 0.2 ) + ( 0.1 ∧ 0.1 ) + ( 0.02 ∧ 0.01 ) + ( 0.07 ∧ 0.04 )\end{matrix}}{\begin{matrix}\begin{matrix}( 0.3 ∨ 0.3 ) + ( 0.32 ∨ 0.35 ) + \\ ( 0.19 ∨ 0.2 ) + ( 0.1 ∨ 0.1 ) +\end{matrix} \\ ( 0.02 ∨ 0.01 ) + ( 0 \text{.07} ∨ 0.04 )\end{matrix}} \times 0.106 = \text{} 0.0978 \qquad ( 10 )$$

A weighted closeness between the to-be-identified fuzzy set U_{X1} and the standard fuzzy set U_{B} is expressed as follows:

$$σ ( {U}_{X 1} , {U}_{B} ) = \frac{\begin{matrix}\begin{matrix}[ ( 0.3 ∧ 0.37 ) + ( 0.32 ∧ 0.41 ) + \\ ( 0.19 ∧ 0.1 ) + ( 0.1 ∧ 0.05 ) +\end{matrix} \\ ( 0.02 ∧ 0 ) + ( 0.07 ∧ 0.07 )\end{matrix}}{\begin{matrix}\begin{matrix}( 0.3 ∨ 0.37 ) + ( 0.32 ∨ 0.41 ) + \\ ( 0.19 ∨ 0.1 ) + ( 0.1 ∨ 0.05 ) +\end{matrix} \\ ( 0.02 ∨ 0 ) + ( 0.07 ∨ 0.07 )\end{matrix}} \times 0.106 = \text{} 0.0768 \qquad ( 11 )$$

A weighted closeness between the to-be-identified fuzzy set U_{X1} and the standard fuzzy set U_{C} is expressed as follows:

$$σ ( {U}_{X 1} , {U}_{C} ) = \frac{\begin{matrix}\begin{matrix}[ ( 0.3 ∧ 0.23 ) + ( 0.32 ∧ 0.47 ) + \\ ( 0.19 ∧ 0.15 ) + ( 0.1 ∧ 0.13 ) +\end{matrix} \\ ( 0.02 ∧ 0.01 ) + ( 0.07 ∧ 0.01 )\end{matrix}}{\begin{matrix}\begin{matrix}( 0.3 ∨ 0.23 ) + ( 0.32 ∨ 0.47 ) + \\ ( 0.19 ∨ 0.15 ) + ( 0.1 ∨ 0.13 ) +\end{matrix} \\ ( 0.02 ∨ 0.01 ) + ( 0.07 ∨ 0.01 )\end{matrix}} \times 0.106 = \text{} 0.0737 . \qquad ( 12 )$$

From the above, it can be seen that σ(U_{X1}, U_{A})>σ(U_{X1}, U_{B})>σ(U_{X1}, U_{C}), that is to say, compared with U_{B} and U_{C,} U_{X1} is closest to U_{A}, which indicates that the potential provenance of the Well X1 is A from the perspective of the light minerals.

Similarly, a weighted closeness between each to-be-identified fuzzy set of wells X2-X12 on the universe U and each of the standard fuzzy sets of provenances A, B and C are calculated, and potential provenances of the wells X2-X12 are determined. The identification results are shown in Table 6.

TABLE 6
Identification results of provenance direction
based on light minerals for each well

| Well | | | | Single-well |
|---|---|---|---|---|
| number | Provenance A | Provenance B | Provenance C | provenance |
| Well | σ (U_{X1}, U_{A}) = | σ (U_{X1}, U_{B}) = | σ (U_{X1}, U_{C}) = | A |
| X1 | 0.0978 | 0.0768 | 0.0737 | |
| Well | σ (U_{X2}, U_{A}) = | σ (U_{X2}, U_{B}) = | σ (U_{X2}, U_{C}) = | B |
| X2 | 0.0885 | 0.0903 | 0.0752 | |
| Well | σ (U_{X3}, U_{A}) = | σ (U_{X3}, U_{B}) = | σ (U_{X3}, U_{C}) = | A |
| X3 | 0.0867 | 0.0783 | 0.0816 | |
| Well | σ (U_{X4}, U_{A}) = | σ (U_{X4}, U_{B}) = | σ (U_{X4}, U_{C}) = | C |
| X4 | 0.0660 | 0.0737 | 0.0978 | |
| Well | σ (U_{X5}, U_{A}) = | σ (U_{X5}, U_{B}) = | σ (U_{X5}, U_{C}) = | C |
| X5 | 0.0768 | 0.0678 | 0.0959 | |
| Well | σ (U_{X6}, U_{A}) = | σ (U_{X6}, U_{B}) = | σ (U_{X6}, U_{C}) = | C |
| X6 | 0.0737 | 0.0752 | 0.0940 | |
| Well | σ (U_{X7}, U_{A}) = | σ (U_{X7}, U_{B}) = | σ (U_{X7}, U_{C}) = | B |
| X7 | 0.0768 | 0.0850 | 0.0783 | |
| Well | σ (U_{X8}, U_{A}) = | σ (U_{X8}, U_{B}) = | σ (U_{X8}, U_{C}) = | C |
| X8 | 0.0833 | 0.0737 | 0.0921 | |
| Well | σ (U_{X9}, U_{A}) = | σ (U_{X9}, U_{B}) = | σ (U_{X9}, U_{C}) = | B |
| X9 | 0.0800 | 0.0850 | 0.0800 | |
| Well | σ (U_{X10}, U_{A}) = | σ (U_{X10}, U_{B}) = | σ (U_{X10}, U_{C}) = | A |
| X10 | 0.0921 | 0.0678 | 0.0800 | |
| Well | σ (U_{X11}, U_{A}) = | σ (U_{X11}, U_{B}) = | σ (U_{X11}, U_{C}) = | C |
| X11 | 0.0800 | 0.0752 | 0.0998 | |
| Well | σ (U_{X12}, U_{A}) = | σ (U_{X12}, U_{B}) = | σ (U_{X12}, U_{C}) = | B |
| X12 | 0.0752 | 0.1018 | 0.0707 | |

Let a universe V={heavy minerals}, which includes six indicators consisting of rutile+zircon+tourmaline, epidote, garnet, sphene, titanomagnetite, and white titanium ore, and the six indicators form a standard fuzzy set {rutile+zircon+tourmaline, epidote, garnet, sphene, titanomagnetite, white titanium ore}. Then a standard fuzzy set of the provenance A on the universe V is: V_{A}={0.4, 0.05, 0.15, 0, 0.40, 0}; a standard fuzzy set of the provenance B on the universe V is: V_{B}={0.28, 0, 0.45, 0, 0.25, 0.02}, and a standard fuzzy set of the provenance C on the universe V is: V_{C}={0.36, 0, 0.26, 0, 0.38, 0}.

For each well, there are six indexes, and a single well has a to-be-identified fuzzy set on the universe V. According to the weighted closeness calculation formula shown in the formula (5), a weighted closeness between each to-be-identified fuzzy set of wells X1-X12 on the universe V and each of the standard fuzzy sets of provenances A, B and C are calculated, and potential provenances of the wells X2-X12 are determined. The identification results are shown in Table 7.

TABLE 7
Identification results of provenance direction
based on heavy minerals for each well

| Well | | | | Single-well |
|---|---|---|---|---|
| number | Provenance A | Provenance B | Provenance C | provenance |
| Well | σ (V_{X1}, V_{A}) = | σ (V_{X1}, V_{B}) = | σ (V_{X1}, V_{C}) = | A |
| X1 | 0.5852 | 0.3123 | 0.4783 | |
| Well | σ (V_{X2}, V_{A}) = | σ (V_{X2}, V_{B}) = | σ (V_{X2}, V_{C}) = | C |
| X2 | 0.4315 | 0.3804 | 0.4498 | |
| Well | σ (V_{X3}, V_{A}) = | σ (V_{X3}, V_{B}) = | σ (V_{X3}, V_{C}) = | C |
| X3 | 0.2228 | 0.4591 | 0.5414 | |
| Well | σ (V_{X4}, V_{A}) = | σ (V_{X4}, V_{B}) = | σ (V_{X4}, V_{C}) = | C |
| X4 | 0.4139 | 0.4406 | 0.4513 | |
| Well | σ (V_{X5}, V_{A}) = | σ (V_{X5}, V_{B}) = | σ (V_{X5}, V_{C}) = | C |
| X5 | 0.4993 | 0.4053 | 0.5293 | |
| Well | σ (V_{X6}, V_{A}) = | σ (V_{X6}, V_{B}) = | σ (V_{X6}, V_{C}) = | B |
| X6 | 0.2984 | 0.4881 | 0.3744 | |
| Well | σ (V_{X7}, V_{A}) = | σ (V_{X7}, V_{B}) = | σ (V_{X7}, V_{C}) = | B |
| X7 | 0.3339 | 0.4227 | 0.3969 | |
| Well | σ (V_{X8}, V_{A}) = | σ (V_{X8}, V_{B}) = | σ (V_{X8}, V_{C}) = | B |
| X8 | 0.3266 | 0.5293 | 0.4053 | |
| Well | σ (V_{X9}, V_{A}) = | σ (V_{X9}, V_{B}) = | σ (V_{X9}, V_{C}) = | B |
| X9 | 0.3266 | 0.5736 | 0.4053 | |
| Well | σ (V_{X10}, V_{A}) = | σ (V_{X10}, V_{B}) = | σ (V_{X10}, V_{C}) = | B |
| X10 | 0.4020 | 0.4881 | 0.3194 | |
| Well | σ (V_{X11}, V_{A}) = | σ (V_{X11}, V_{B}) = | σ (V_{X11}, V_{C}) = | C |
| X11 | 0.4783 | 0.4415 | 0.5852 | |
| Well | σ (V_{X12}, V_{A}) = | σ (V_{X12}, V_{B}) = | σ (V_{X12}, V_{C}) = | B |
| X12 | 0.2664 | 0.5971 | 0.4315 | |

Let a universe Y={trace elements}, which includes nine indicators consisting of Sc, V, Cr, Co, Ni, Zn, Ga, Rb, and Zr, and the nine indicators form a standard fuzzy set {Sc, V, Cr, Co, Ni, Zn, Ga, Rb, Zr}. Then a standard fuzzy set of the provenance A on the universe Y is: Y_{A}={0.12, 0.05, 0.17, 0.2, 0.01, 0.28, 0.1, 0.04, 0.03}; a standard fuzzy set of the provenance A on the universe Y is: Y_{B}={0.2, 0.11, 0.32, 0.05, 0.16, 0.02, 0.07, 0.05, 0.02}; and a standard fuzzy set of the provenance A on the universe Y is: Y_{C}={0.32, 0.2, 0.23, 0.06, 0.03, 0.11, 0.01, 0.04, 0}.

For each well, there are nine indexes, and a single well has a to-be-identified fuzzy set on the universe Y. According to the weighted closeness calculation formula shown in the formula (5), a weighted closeness between each to-be-identified fuzzy set of wells X1-X12 on the universe Y and each of the standard fuzzy sets of provenances A, B and C are calculated, and potential provenances of the wells X2-X12 are determined. The identification results are shown in Table 8.

TABLE 8
Identification results of provenance direction
based on trace elements for each well

| Well | | | | Single-well |
|---|---|---|---|---|
| number | Provenance A | Provenance B | Provenance C | provenance |
| Well | σ (Y_{X1}, Y_{A}) = | σ (Y_{X1}, Y_{B}) = | σ (Y_{X1}, Y_{C}) = | A |
| X1 | 0.2449 | 0.0962 | 0.1011 | |
| Well | σ (Y_{X2}, Y_{A}) = | σ (Y_{X2}, Y_{B}) = | σ (Y_{X2}, Y_{C}) = | B |
| X2 | 0.1062 | 0.1770 | 0.1527 | |
| Well | σ (Y_{X3}, Y_{A}) = | σ (Y_{X3}, Y_{B}) = | σ (Y_{X3}, Y_{C}) = | C |
| X3 | 0.1339 | 0.1252 | 0.1369 | |
| Well | σ (Y_{X4}, Y_{A}) = | σ (Y_{X4}, Y_{B}) = | σ (Y_{X4}, Y_{C}) = | B |
| X4 | 0.1114 | 0.1662 | 0.1339 | |
| Well | σ (Y_{X5}, Y_{A}) = | σ (Y_{X5}, Y_{B}) = | σ (Y_{X5}, Y_{C}) = | C |
| X5 | 0.1141 | 0.1369 | 0.2043 | |
| Well | σ (Y_{X6}, Y_{A}) = | σ (Y_{X6}, Y_{B}) = | σ (Y_{X6}, Y_{C}) = | B |
| X6 | 0.0844 | 0.0858 | 0.0650 | |
| Well | σ (Y_{X7}, Y_{A}) = | σ (Y_{X7}, Y_{B}) = | σ (Y_{X7}, Y_{C}) = | C |
| X7 | 0.0867 | 0.1594 | 0.1698 | |
| Well | σ (Y_{X8}, Y_{A}) = | σ (Y_{X8}, Y_{B}) = | σ (Y_{X8}, Y_{C}) = | A |
| X8 | 0.1628 | 0.1088 | 0.1196 | |
| Well | σ (Y_{X9}, Y_{A}) = | σ (Y_{X9}, Y_{B}) = | σ (Y_{X9}, Y_{C}) = | A |
| X9 | 0.1281 | 0.0937 | 0.0733 | |
| Well | σ (Y_{X10}, Y_{A}) = | σ (Y_{X10}, Y_{B}) = | σ (Y_{X10}, Y_{C}) = | B |
| X10 | 0.0777 | 0.0962 | 0.0691 | |
| Well | σ (Y_{X11}, Y_{A}) = | σ (Y_{X11}, Y_{B}) = | σ (Y_{X11}, Y_{C}) = | B |
| X11 | 0.1339 | 0.1456 | 0.1339 | |
| Well | σ (Y_{X12}, Y_{A}) = | σ (Y_{X12}, Y_{B}) = | σ (Y_{X12}, Y_{C}) = | B |
| X12 | 0.0986 | 0.2352 | 0.1431 | |

According to Table 6 through Table 8, during the H sedimentary period, the lake basin E was characterized by multi-provenance supply as a whole, and the sediments in the lake basin E were input from three directions: the paleocontinent A in the northwest, the paleocontinent B in the north and the paleocontinent C in the south. Among the 12 wells in the study area, the well X1, the well X5 and the well X12 have a single-direction provenance supply; the well X2, the well X3, the well X4, the well X6, the well X7, the well X9, the well X10 and the well X11 have two-way provenance supply; and the well X8 has three-direction provenance supply.

In order to know more clearly the supply relationship between the wells with two-way and three-direction provenance supply and each provenance, an embodiment of the disclosure also includes the following steps: summing weighted closenesses of wells with two-way and three-direction provenance supply to obtain a comprehensive weighted closeness of each well; according to the comprehensive weighted closeness of each well, a primary provenance, a relatively primary provenance and a secondary provenance of the target horizon are determined according to the principle of proximity selection (a provenance with the largest closeness to a well is the primary provenance, a provenance with the middle closeness to the well is the relatively primary provenance, and a provenance with the smallest closeness to the well is the secondary provenance). The results are shown in Table 9.

TABLE 9
Comprehensive identification results of provenance direction of each well

| | | | | | Relatively | |
|---|---|---|---|---|---|---|
| Well | Provenance | Provenance | Provenance | Primary | primary | Secondary |
| number | A | B | C | provenance | provenance | provenance |
| Well | / | / | / | A | / | / |
| X1 | | | | | | |
| Well | / | σ(U + V + Y) = | σ(U + V + Y) = | C | B | / |
| X2 | | 0.6477 | 0.6777 | | | |
| Well | σ(U + V + Y) = | / | σ(U + V + Y) = | C | A | / |
| X3 | 0.4434 | | 0.7599 | | | |
| Well | / | σ(U + V + Y) = | σ(U + V + Y) = | C | B | / |
| X4 | | 0.6805 | 0.6831 | | | |
| Well | / | / | / | C | / | / |
| X5 | | | | | | |
| Well | / | σ(U + V + Y) = | σ(U + V + Y) = | B | C | / |
| X6 | | 0.6491 | 0.5334 | | | |
| Well | / | σ(U + V + Y) = | σ(U + V + Y) = | B | C | / |
| X7 | | 0.6671 | 0.6450 | | | |
| Well | σ(U + V + Y) = | σ(U + V + Y) = | σ(U + V + Y) = | B | C | A |
| X8 | 0.5727 | 0.7118 | 0.6170 | | | |
| Well | σ(U + V + Y) = | σ(U + V + Y) = | / | B | A | / |
| X9 | 0.5347 | 0.7523 | | | | |
| Well | σ(U + V + Y) = | σ(U + V + Y) = | / | B | A | / |
| X10 | 0.5718 | 0.6521 | | | | |
| Well | / | σ(U + V + Y) = | σ(U + V + Y) = | C | B | / |
| X11 | | 0.6623 | 0.8189 | | | |
| Well | / | / | / | B | / | / |
| X12 | | | | | | |

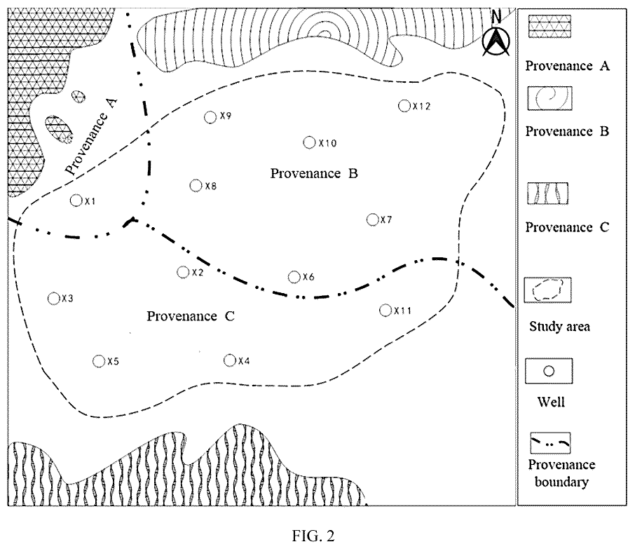

According to the above results, a plane distribution view of the main provenances in the study area shown in FIG. 2 is drawn. From FIG. 2, a main provenance of each well can be known clearly and intuitively.

In summary, the present disclosure takes into account the characteristics of heavy mineral content, light mineral content, and trace element content comprehensively, resulting in higher accuracy of the outcomes. Moreover, by employing fuzzy mathematical methods, the disclosure enables the quantification of provenance identification. Compared to existing technologies, the present disclosure represents a significant advancement.

The above description is merely an exemplary embodiment of the present disclosure, and is not intended to limit the disclosure in any form. Although the disclosure has been described in detail with a preferred embodiment, it is not intended to limit the disclosure. Any person skilled in the art, within the scope of the technical solution of the disclosure, may make some changes or modifications to the technical content revealed above to create equivalent embodiments that are equivalent variations. However, any simple modifications, equivalent variations, and modifications made to the above embodiments that do not depart from the content of the technical solution of the disclosure, based on the technical essence of the disclosure, are still within the scope of the technical solution of the disclosure.

1. A quantitative identification method for provenance of clastic rocks, comprising the following steps:
S1, determining a study area, a target horizon of the study area and potential provenances of the target horizon, and performing sampling on known wells of the target horizon and the potential provenances to obtain rock samples;
S2, measuring contents of light minerals, heavy minerals, and trace elements in the rock samples to obtain measurement results of the known wells and measurement results of the potential provenances, establishing standard fuzzy sets based on the measurement results of the potential provenances, and establishing to-be-identified fuzzy sets based on the measurement results of the known wells;
S3, assigning weight coefficients related to importance in provenance discrimination to three indicators respectively corresponding to the light minerals, the heavy minerals and the trace elements;
S4, calculating weighted closenesses between the to-be-identified fuzzy sets and the standard fuzzy sets according to the weight coefficients; and
S5, determining a provenance of the target horizon according to the weighted closenesses and a principle of proximity selection;
wherein after the provenance of the target horizon is determined, the quantitative identification method for provenance of clastic rocks further comprises:
in response to a ratio of sedimentary rock debris of a parent rock of the provenance being greater than a preset threshold, exploring and developing the target horizon to obtain oil and gas from the target horizon.

2. The quantitative identification method for provenance of clastic rocks as claimed in claim 1, wherein the measuring contents of light minerals, heavy minerals, and trace elements in the rock samples in step S2 comprises:
performing thin-section preparation and microscopic identification on the rock samples to thereby obtain the contents of the light minerals and the contents of the heavy minerals; and
performing content analysis on the trace elements by plasma mass spectrometry, to thereby obtain the contents of the trace elements.

3. The quantitative identification method for provenance of clastic rocks as claimed in claim 1, wherein in step S2, the light minerals comprise quartz, feldspar, rock debris, and other light minerals, and the rock debris comprises igneous rock debris, metamorphic rock debris, and sedimentary rock debris;
wherein the heavy minerals comprise heavy mineral assemblage, epidote, garnet, sphene, titanomagnetite and white titanium ore, and the heavy mineral assemblage consists of rutile, zircon and tourmaline; and
wherein the trace elements comprise scandium (Sc), vanadium (V), chromium (Cr), cobalt (Co), nickel (Ni), zinc (Zn), gallium (Ga), rubidium (Rb) and zirconium (Zr).

4. The quantitative identification method for provenance of clastic rocks as claimed in claim 1, wherein the assigning weight coefficients related to importance in provenance discrimination to three indicators respectively corresponding to the light minerals, the heavy minerals and the trace elements in step S3 comprises:
using a pairwise comparison method to assign the weight coefficients to the three indicators.

5. The quantitative identification method for provenance of clastic rocks as claimed in claim 4, wherein the using a pairwise comparison method to assign the weight coefficients to the three indicators comprises:
step S31, based on geological experience, performing pairwise comparison on importance in provenance discrimination of the three indicators respectively corresponding to the light minerals, the heavy minerals, and the trace elements, to thereby construct a judgment matrix:
$$A = ( \begin{matrix}1 & \frac{1}{5} & \frac{1}{3} \\ 5 & 1 & 3 \\ 3 & \frac{1}{3} & 1\end{matrix} ) ; \qquad ( 1 )$$
step S32, normalizing each column of the judgment matrix to obtain a a first matrix A₁, expressed as follows:
$${A}_{1} = ( \begin{matrix}\frac{1}{9} & \frac{3}{23} & \frac{1}{13} \\ \frac{5}{9} & \frac{15}{23} & \frac{9}{23} \\ \frac{3}{9} & \frac{5}{23} & \frac{3}{13}\end{matrix} ) ; \qquad ( 2 )$$
step S33, performing a summation operation on the first matrix A₁ to obtain a second matrix A₂, expressed as follows:
$${A}_{2} = ( \begin{matrix}\frac{857}{2691} \\ \frac{5113}{2691} \\ \frac{2103}{2691}\end{matrix} ) ; \qquad ( 3 )$$
step S34, normalizing the second matrix A₂ to obtain a third matrix A₃, expressed as follows:
$${A}_{3} = ( \begin{matrix}0.106 \\ 0.634 \\ 0.26\end{matrix} ) , \qquad ( 4 )$$
and
based on the third matrix A₃, determining the weight coefficients related to importance in provenance discrimination for the three indicators to be 0.106 for the light minerals, 0.634 for the heavy minerals, and 0.260 for the trace elements.

6. The quantitative identification method for provenance of clastic rocks as claimed in claim 1, wherein in step S4, each of the weighted closenesses is calculated through the following formula:
$$σ ( A , B ) {=}^{def} \frac{{\sum }_{k = 1}^{n} [ A ( {x}_{k} ) ∧ B ( {x}_{k} ) ]}{{\sum }_{k = 1}^{n} [ A ( {x}_{k} ) ∨ B ( {x}_{k} ) ]} \times q \qquad ( 5 )$$
where σ(A, B) represents a weighted closeness between a set A and a set B; the set A and the set B represent a to-be-identified fuzzy set and a standard fuzzy set, respectively; n represents a total number of elements in each of the to-be-identified fuzzy set and the standard fuzzy set; xₖ represents a k-th element in a corresponding fuzzy set; ∧ represents taking an infimum when two related elements are compared; ∨ represents taking a supremum when two related elements are compared; and q represents a corresponding weight coefficient.

7. The quantitative identification method for provenance of clastic rocks as claimed in claim 1, wherein when the provenance of the target horizon comprises multiple provenances, the quantitative identification method for provenance of clastic rocks further comprises:
step S6, summing weighted closenesses of light minerals, heavy minerals and trace elements of each provenance of the multiple provenances to obtain a comprehensive weighted closeness of each provenance of the multiple provenances; and
step S7, determining a primary provenance, a relatively primary provenance and a secondary provenance of the target horizon according to the comprehensive weighted closeness of each provenance of the multiple provenances and the principle of proximity selection.

8. The quantitative identification method for provenance of clastic rocks as claimed in claim 1, wherein the parent rock consists of the sedimentary rock debris, igneous rock debris, and metamorphic rock debris, and the quantitative identification method for provenance of clastic rocks further comprises: when a ratio of the igneous rock debris and the metamorphic rock debris to the sedimentary rock debris is greater than a second threshold, determining that a storage capacity of the target horizon is not optimal and determining not to develop the target horizon.

9. A quantitative identification method for provenance of clastic rocks, comprising the following steps:
S1, determining a study area, a target horizon of the study area and potential provenances of the target horizon, and performing sampling on known wells of the target horizon and the potential provenances to obtain rock samples;
S2, measuring contents of light minerals, heavy minerals, and trace elements in the rock samples to obtain measurement results of the known wells and measurement results of the potential provenances, establishing standard fuzzy sets based on the measurement results of the potential provenances, and establishing to-be-identified fuzzy sets based on the measurement results of the known wells;
S3, assigning weight coefficients related to importance in provenance discrimination to three indicators respectively corresponding to the light minerals, the heavy minerals and the trace elements;
S4, calculating weighted closenesses between the to-be-identified fuzzy sets and the standard fuzzy sets according to the weight coefficients;
S5, determining provenances of the known wells of the target horizon according to the weighted closenesses and a principle of proximity selection; and
S6, determining a ratio of sedimentary rock debris of a parent rock of each of the provenances of the known wells of the target horizon to igneous rock debris and metamorphic rock debris of the parent rock of each of the provenances of the known wells of the target horizon, and preferentially exploring and developing of a target well with a highest ratio of sedimentary rock debris of the known wells of the target horizon of the study region to obtain oil and gas from the target horizon.

10. A quantitative identification method for provenance of clastic rocks, comprising the following steps:
S1, determining a study area, a target horizon of the study area and potential provenances of the target horizon, and performing sampling on known wells of the target horizon and the potential provenances to obtain rock samples;
S2, measuring contents of light minerals, heavy minerals, and trace elements in the rock samples to obtain measurement results of the known wells and measurement results of the potential provenances, establishing standard fuzzy sets based on the measurement results of the potential provenances, and establishing to-be-identified fuzzy sets based on the measurement results of the known wells;
S3, assigning weight coefficients related to importance in provenance discrimination to three indicators respectively corresponding to the light minerals, the heavy minerals and the trace elements;
S4, calculating weighted closenesses between the to-be-identified fuzzy sets and the standard fuzzy sets according to the weight coefficients;
S5, determining a provenance of the target horizon according to the weighted closenesses and a principle of proximity selection; and
S6, using the provenance of the target horizon to perform reservoir sandstone body prediction of the target horizon and guiding oil and gas exploration deployment based on the reservoir sandstone body prediction.
